(19) United States
(12) Patent Application Publication
(10) Pub. No.: US 2013/0130934 A1
(43) Pub. Date: May 23, 2013

(54) EFFICIENT HIGH-THROUGHPUT SCREEN FOR IDENTIFYING NOVEL CHEMICAL COMPOUNDS WHICH DISRUPT THE FUNGAL VACUOLE

(71) Applicant: Board of Supervisors of Louisiana State University and Agriculture and Mechanical College, Baton Rouge, LA (US)

(72) Inventor: Glen Edwin Palmer, New Orleans, LA (US)

(73) Assignee: Board of Supervisors of Louisiana State University and agricultural and Mechanical College, Baton Rouge, LA (US)

(21) Appl. No.: 13/658,950

(22) Filed: Oct. 24, 2012

Related U.S. Application Data
(60) Provisional application No. 61/561,069, filed on Nov. 17, 2011.

(51) Int. Cl. C40B 30/06; C12Q 1/18

(52) U.S. Cl. 506/10; 435/32

(57) ABSTRACT

A high-throughput screening method for identifying new anti-fungal compounds, including: growing an adenine-requiring mutant fungus under conditions to induce bioaccumulation of P-ribosylaminoimidazole; treating the mutant fungus with chemical compounds; and determining if the chemical compounds cause white discoloration of the mutant fungus. A method to determine the effective concentration of an anti-fungal. A method to detect chemical disruption of the fungal vacuole of a fungus.

### CROSS-REFERENCE TO RELATED APPLICATIONS

This application claims benefit of U.S. Provisional Application 61/561,069 filed Nov. 17, 2011, the entire disclosure of which is incorporated herein by reference.

### FIELD

The present invention relates to methods of identifying therapeutic compounds, and in particular though non-limiting embodiments, to methods of identifying compounds with antifungal properties.

### BACKGROUND

Recent decades have seen dramatic increases in the incidence of mucosal and disseminated mycoses. Mycoses are common and various physiological and environmental conditions can contribute to the development of a fungal disease. Taking antibiotics for lengthy periods of time, weakened immune systems, diabetes, steroid therapies, trauma and age can all increase susceptibility to fungal infections.

Mycoses may be classified based upon the tissue levels initially infected. Superficial mycoses are limited infections affecting the outermost layer of skin and hair only. Cutaneous mycoses extend deeper into the epidermis and may invoke host immune responses. A common example of a cutaneous mycosis is athlete's foot. Subcutaneous mycoses involve the dermis, subcutaneous tissues, muscle and fascia. These infections are chronic and are difficult to treat. Systemic mycoses are infections that may spread to numerous organ systems. Systemic mycoses may result from virulent organisms or from opportunistic pathogens in patients with immune deficiencies. Opportunistic mycoses include Candidiasis, Crtyptococcosis and Aspergillosis.

Antifungal medication is used for a variety of applications, and the size of the market reflects the continued demand for these products. According to GBI Research, the global market for antifungals in 2009 was estimated to be $7.4 billion and is expected to grow at a Compound Annual Growth Rate (CAGR) of 1.9% to record sales of $8.4 billion by 2016. These market trends tend to reveal that in the coming years interest in antifungal agents will continue to increase, resulting in a need for continued innovation.

Various antifungal drugs are currently used to treat fungal infections. Generally, antifungals work by addressing differences between mammalian cells and fungal cells; however, because fungi and mammals are eukaryotes, mammalian cells and fungal cells are more similar than bacteria and mammalian cells. The most commonly used antifungals are azoles, polyenes, and caspofungin. Azoles inhibit synthesis of ergosterol, which is necessary for normal fungal membrane function and depletion leads to inhibition of fungal growth. Polyenes disrupt fungal membrane function by binding with sterols, including ergosterol. Caspofungin inhibits fungal cell wall synthesis.

Unfortunately, the most widely used antifungal treatments also have serious limitations including host toxicity, limited formulations, and/or the emergence of resistant fungal isolates. Another major limitation of current antifungal treatments is that they only act upon a narrow range of fungal components. Currently, there are a plethora of antifungal medications for treating fungal infections ranging from athlete's foot to systemic infections affecting the immunocompromised that are caused by a variety of species such as Candida albicans. Nonetheless, only a few antifungals are broadly effective and most cause extensive side-effects and eventually result in problems of fungal resistance. As such, there is an urgent need for effective new antifungals with low host toxicity and availability in a range of formulations. Furthermore, new antifungals will be of increasing importance as resistance to existing antifungals becomes more prevalent.

Due to the fact that greater than 98% of medicines at the preclinical level are terminated prior to their utilization in humans, it is seen as an advantage by companies to possess a mechanism in which several drugs can be identified from one platform screening tool. Therefore, there is an urgent and unmet need for methods of identifying novel therapeutic compounds with potent antifungal properties, which offers the potential for a number of potentially commercializable therapeutics.

### SUMMARY

In an example embodiment of the present disclosure, a method of screening a chemical compound for antifungal properties is provided, including: growing an adenine-requiring mutant fungus under conditions to induce bioaccumulation of P-ribosylaminoimidazole; treating the mutant fungus with the chemical compound; and determining if the chemical compound causes at least one of white discoloration of the mutant fungus and secretion of P-ribosylaminoimidazole. The mutant fungus may be one of Candida albicans, Candida glabrata, Saccharomyces cerevisiae, and Cryptococcus neoformans. The mutant fungus may be an ade1 mutant or it may be an ade2 mutant. The mutant fungus may be grown in a well of a microwell plate. The chemical compound may be added to the well of the microwell plate.

In an example embodiment of the present disclosure, a method of simultaneously screening multiple chemical compounds for antifungal properties is provided, including: growing multiple cultures of an adenine-requiring mutant fungus under conditions to induce bioaccumulation of P-ribosylaminoimidazole; treating each of the multiple cultures of the mutant fungus with one of the multiple chemical compounds; and identifying each of the multiple chemical compounds that cause at least one of white discoloration of the mutant fungus and secretion of P-ribosylaminoimidazole.

In an example embodiment of the present disclosure, a method of determining an effective concentration of an antifungal compound is provided, including: growing an adenine-requiring mutant fungus under conditions to induce bioaccumulation of P-ribosylaminoimidazole; treating the mutant fungus with a variety concentrations of the antifungal compound; and identifying a minimum concentration required to produce at least one of white discoloration of the mutant fungus and secretion of P-ribosylaminoimidazole. The mutant fungus may be grown in more than one well of a microwell plate. One of the variety of concentrations of the antifungal compound may be added to each of the more than one well of the microwell plate.

In an example embodiment of the present disclosure, a method for screening multiple chemical compounds that target the fungal vacuole is provided, including: growing an adenine-requiring mutant fungus under conditions to induce bioaccumulation of P-ribosylaminoimidazole; treating the mutant fungus with the multiple chemical compounds; and identifying each of the multiple chemical compounds that cause at least one of white discoloration of the mutant fungus and secretion of P-ribosylaminoimidazole. The each of the multiple chemical compounds that cause at least one of white discoloration of the mutant fungus and secretion of P-ribosylaminoimidazole may be further tested for anti-mammalian lysosome properties.

### BRIEF DESCRIPTION OF DRAWINGS

FIG. 1 is a representative drawing of a fungus cell showing certain cellular structures.

FIG. 2 is a flow diagram of the adenine pathway of non-mutant fungus, according to an exemplary embodiment of the present invention.

FIG. 3 is a top view of a 96-well microwell plate, according to an exemplary embodiment of the present invention.

FIG. 4 shows Candida albicans ade2 with red pigmentation in a strain with normal vacuolar function and without red pigmentation in a strain with disrupted vacuolar function, according to an exemplary embodiment of the present invention.

FIG. 5 is representative drawing demonstrating pigment secretion, according to an exemplary embodiment of the present invention.

FIG. 6 shows test results for a chemical compound administered at various concentrations, according to an exemplary embodiment of the present invention.

### DESCRIPTION

Embodiments of the present disclosure provide a high-throughput screening method for identifying new anti-fungal compounds. In certain embodiments, the present invention allows for the identification of anti-fungal compounds by detecting color changes in mutant strains of fungi. In other embodiments, the present invention allows for identification of antifungal compounds by detecting secretion of pigments. In still other embodiments, the present invention provides a method of determining the effective concentration of an anti-fungal. In various embodiments, the present invention may be used to detect disruption of the fungal vacuole of a fungus. Certain embodiments may be used to identify potential therapeutic compounds that disrupt the mammalian lysosome, which may be potential anti-cancer compounds.

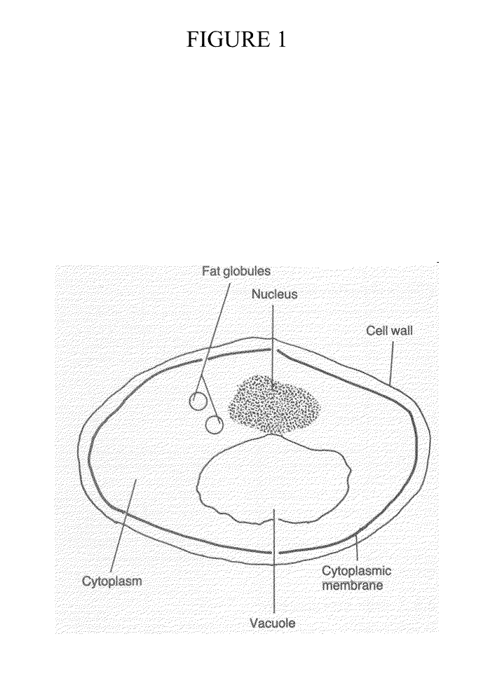

A fungal vacuole is an acidic compartment containing a variety of hydrolytic enzymes. See FIG. 1. The fungal vacuole serves a number of vital functions in normal fungal biology, including stress tolerance (oxidative, osmotic, pH, starvation, detoxification—allows for survival in host environment), macromolecule degradation, and storage (amino acids and metal ions). The fungal vacuole has also been shown to support polarized hyphal growth, a critical component of Candida albicans virulence. The fungal vacuole is required for Candida albicans pathogenesis and is required to support host colonization and infection by Cryptococcus neoformans, the causative agent of lethal meningoencephalitis.

The fungal vacuole and its role in the biology and pathogenesis of fungi create potential as a new anti-fungal target. Vacuoles are ubiquitous throughout fungi creating the possibility to broad-spectrum application for an anti-fungal disrupting the functions of the vacuole. Disruption or rupture of the vacuole may inhibit infection, survival or even cause autolysis. The fungal vacuole is analogous to the mammalian lysosome; vacuoles are not found in mammalian cells. Because the fungal vacuole is a distinct cellular component of fungi and is a necessary component for both survival and pathogenesis, chemical compounds that exhibit anti-fungal properties through targeting of the fungal vacuole are a distinct mechanism from conventional therapies and are less likely to be toxic to mammalian cells. Moreover, cross-resistance with other antifungal agents is less likely to occur. This is a shift from the current paradigm for anti-fungal therapies.

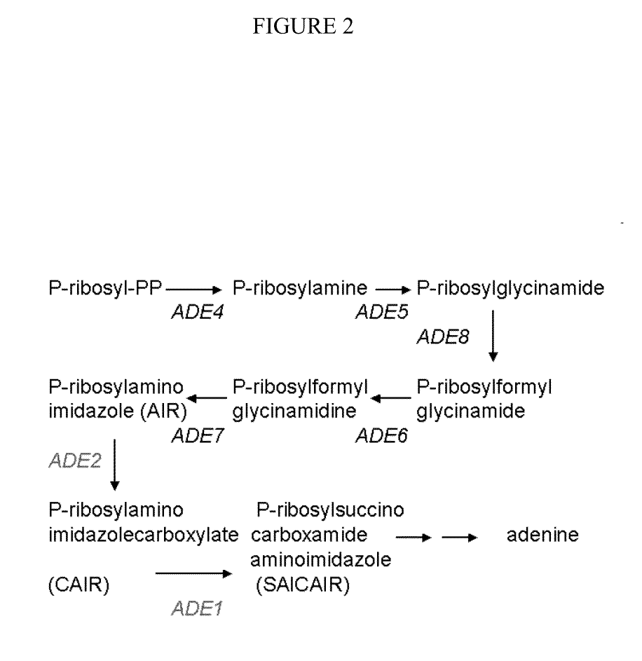

Certain mutant strains of fungi, deficient in enzymes of the adenine synthesis pathway (FIG. 2), accumulate a red pigment under certain growth conditions. Fungi generally produce adenine through the adenine pathway shown in FIG. 2. In the event of a ade1 mutation or a ade2 mutation, the mutated fungus will accumulate large amounts of P-ribosylaminoimidazole (AIR). AIR is a red pigmented biosynthetic intermediate that normally accumulates in the vacuole of the mutant fungus. The accumulation of red pigmentation in the fungus may then be used to determine if the fungal vacuole is intact. If the vacuolar function is disrupted, AIR will not accumulate in the vacuole and the fungal sample will appear white instead of red. Alternatively, if the vacuolar function is disrupted, AIR pigment may be secreted from the fungal cell, which secreted AIR pigment may be detected in a growth medium.

According to exemplary embodiments of the present disclosure, an assay for screening for antifungals that target the fungal vacuole is provided. In example embodiments, the assay identifies new effective drugs based on the particular physiology of the vacuole. In further embodiments, the assay identifies new effective drugs, due to the particular physiology of the vacuole vis-à-vis yeast biology.

According to exemplary embodiments of the present disclosure, certain mutant varieties of pathogenic fungi, such as Candida albicans and Candida glabrata, and non-pathogenic fungi, such as Saccharomyces cerevisiae, undergo a robust phenotypic color change under certain culture conditions. Specifically, the ade1 and ade2 mutant varieties of these fungal pathogens produce and accumulate AIR when the fungal vacuole is functioning normally; whereas the fungus will turn white if the function or integrity of the vacuole is disrupted. In example embodiments of the present disclosure, taking advantage of this detectable color change, chemical compounds that specifically disrupt the fungal vacuole may be identified. Because the vacuole performs vital functions in normal fungal biology targeted disruption of the fungal vacuole provides a powerful new approach to identifying new anti-fungal agents. Furthermore, the vacuole is an attractive target for anti-fungals because the equivalent organelle in mammalian cells, the lysosome, has diverged in terms of its cellular functions and molecular composition from the fungal vacuole. Thus, chemical compounds that exhibit anti-fungal properties through specific targeting of the fungal vacuole are less likely to be toxic to mammalian cells. However, chemical compounds identified through the chemical screen which lack fungal specific activity and thus disrupt the mammalian lysosome, may also be used as a therapeutic in treating a variety of human diseases including cancer. Thus the described screen may yield therapeutic agents for cancer in addition to the anticipated antifungal drugs.

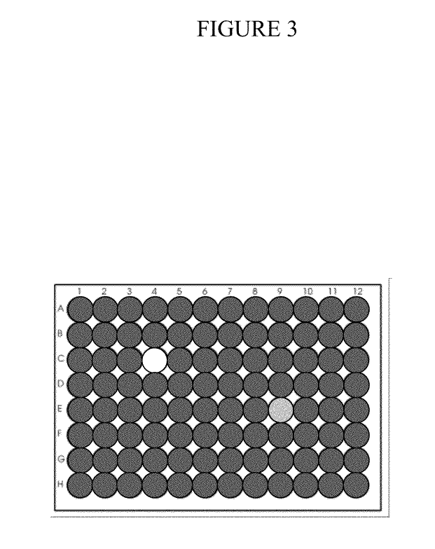

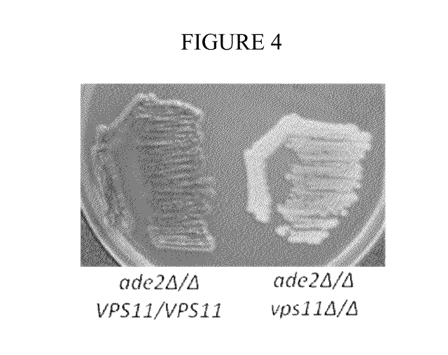

In example embodiments of the present disclosure, an ade2 mutant high-throughput screen includes growing ade2 mutant yeast or filamentous fungi in culture dishes such as 96-well assay plates. See FIG. 3. In further embodiments, the yeast are grown under conditions known in the art to induce red-pigment bioaccumulation of AIR. Also, according to example embodiments, various chemical compounds derived from commercially-available small molecule libraries or other molecule collections may be added to the yeast culture wells, which are grown until the pigment is detectable. In exemplary embodiments, those chemical compounds that disrupt the fungal vacuole are identified by the white coloration of the fungi in the respective well of the plate (i.e. loss of pigmentation). By contrast, chemical agents that are ineffective at disrupting the vacuole do not cause a change in the red pigmentation of the fungus. FIG. 4 shows a strains of mutant fungus where the fungus appears red under normal vacuole function and appears white when vacuole function is disrupted.

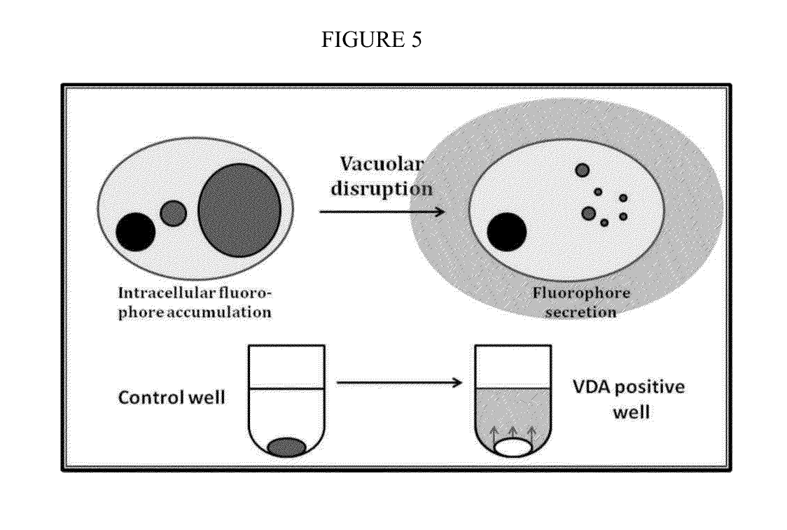

In example embodiments of the present disclosure, antifungal compounds may be discovered by identifying compounds that cause secretion of AIR from a mutant fungus. FIG. 5 is a representative drawing showing accumulation of AIR within the mutant fungus cell under normal vacuolar function and AIR secretion when vacuolar function is disrupted, according to an example embodiment of the present disclosure.

In example embodiments of the present disclosure, use of the screening method yields a new class of broad-spectrum antifungal compounds. According to exemplary embodiments, the compounds specifically target the fungal vacuole and therefore have a mechanism of action distinct from existing antifungals. In further embodiments, the antifungal compounds may be developed into clinically-applicable therapies, providing new treatment options for a wide variety of medical mycoses.

In further embodiments of the present disclosure, the high-throughput screen is used to identify novel therapeutic compounds with potent antifungal properties.

According to example embodiments of the present disclosure, the high-throughput chemical screen applies different chemical agents to ade2 yeast in order to visualize and confirm that the chemical agents proximately cause the disruption or destruction of the vacuole. According to further embodiments, the drug screen utilizes chemical agents to strategically target the vacuole.

In further embodiments of the present disclosure, the screen identifies drugs that specifically disrupt the fungal vacuole.

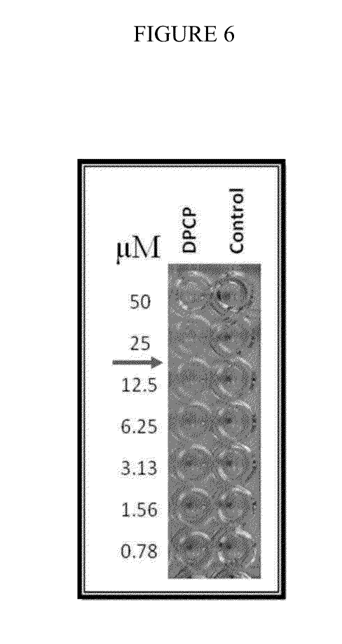

In still further embodiments, the present disclosure provides a method of determining an effective concentration of an anti-fungal compound. For a compound having either known or unknown anti-fungal properties, various concentrations may be applied to cultures of an adenine-requiring mutant fungus, which may be ade2 Candida albicans. The cultures are grown under conditions known to induce bioaccumulation of AIR. The cultures may be grown in a microwell plate or any other culture means wherein various concentrations may be tested. The effective concentration is determined by reviewing the lowest concentration of the anti-fungal to cause white discoloration of the mutant fungus or secretion of the AIR pigment from the mutant fungus. FIG. 6 shows test results wherein an antifungal agent and a control where administered at various concentrations. The minimum concentration to cause a white (non-red) discoloration represents an effective concentration of that agent for antifungal purposes.

While the embodiments of the present disclosure are described herein with reference to various implementations and exploitations, it will be understood that these embodiments are illustrative and that the scope of the inventions is not limited to them. Many variations, modifications, additions, and improvements are possible, including the application of a similar screen to different species of fungi. Further still, any steps described herein may be carried out in any desired order, and any desired steps may be added or deleted.

What is claimed:

1. A method of screening a chemical compound for antifungal properties, comprising: growing an adenine-requiring mutant fungus under conditions to induce bioaccumulation of P-ribosylaminoimidazole; treating the mutant fungus with the chemical compound; and determining if the chemical compound causes at least one of white discoloration of the mutant fungus and secretion of P-ribosylaminoimidazole.

2. The method of claim 1, wherein the mutant fungus is one of Candida albicans, Candida glabrata, Saccharomyces cerevisiae, and Cryptococcus neoformans.

3. The method of claim 1, wherein the mutant fungus is an ade1 mutant.

4. The method of claim 1, wherein the mutant fungus is an ade2 mutant.

5. The method of claim 1, wherein the mutant fungus is grown in a well of a microwell plate.

6. The method of claim 5, wherein the chemical compound is added to the well of the microwell plate.

7. A method of simultaneously screening multiple chemical compounds for antifungal properties, comprising: growing multiple cultures of an adenine-requiring mutant fungus under conditions to induce bioaccumulation of P-ribosylaminoimidazole; treating each of the multiple cultures of the mutant fungus with one of the multiple chemical compounds; and identifying each of the multiple chemical compounds that cause at least one of white discoloration of the mutant fungus and secretion of P-ribosylaminoimidazole.

8. The method of claim 7, wherein the mutant fungus is one of Candida albicans, Candida glabrata, Saccharomyces cerevisiae, and Cryptococcus neoformans.

9. The method of claim 7, wherein the mutant fungus is an ade1 mutant.

10. The method of claim 7, wherein the mutant fungus is an ade2 mutant.

11. A method of determining an effective concentration of an antifungal compound, comprising: growing an adenine-requiring mutant fungus under conditions to induce bioaccumulation of P-ribosylaminoimidazole; treating the mutant fungus with a variety concentrations of the antifungal compound; and identifying a minimum concentration required to produce white discoloration of the mutant fungus.

12. The method of claim 11, wherein the mutant fungus is one of Candida albicans, Candida glabrata, Saccharomyces cerevisiae, and Cryptococcus neoformans.

13. The method of claim 12, wherein the mutant fungus is an ade1 mutant.

14. The method of claim 12, wherein the mutant fungus is an ade2 mutant.

15. The method of claim 11, wherein the mutant fungus is grown in more than one well of a microwell plate.

16. The method of claim 15, wherein one of the variety of concentrations of the antifungal compound is added to each of the more than one well of the microwell plate.

17. A method for screening multiple chemical compounds that target the fungal vacuole, comprising: growing an adenine-requiring mutant fungus under conditions to induce bioaccumulation of P-ribosylaminoimidazole; treating the mutant fungus with the multiple chemical compounds; and identifying each of the multiple chemical compounds that cause at least one of white discoloration of the mutant fungus and secretion of P-ribosylaminoimidazole.

18. The method of claim 17, wherein the each of the multiple chemical compounds that cause at least one of white discoloration of the mutant fungus and secretion of P-ribosylaminoimidazole are further tested for anti-mammalian lysosome properties.
